(19) United States
(12) Patent Application Publication
(10) Pub. No.: US 2008/0255375 A1
(43) Pub. Date: Oct. 16, 2008

(54) Alkenylsuccinic Anhydrides Formed From Oligomers Of C4-To C8-Olefins And Maleic Anhydride, Processes For Their Preparation And Their Use

(75) Inventors: Arno Lange, Bad Durkheim (DE); Szilard Csihony, Weinheim (DE); Wolfgang Gaschler, Ludwigshafen (DE); Markus Schmid, Deidesheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF AKTIENGESELLSCHAFT, LUDWIGSHAFEN (DE)

(21) Appl. No.: 12/092,770

(22) PCT Filed: Nov. 13, 2006
(86) PCT No.: PCT/EP06/68375

§ 371 Date: May 6, 2008

(30) Foreign Application Priority Data
Nov. 18, 2005 (DE) 10-2005-055-541.1

(51) Int. Cl. C07C 57/02

(52) U.S. Cl. 549/255

(57) ABSTRACT

Alkenylsuccinic anhydrides obtainable by reaction of maleic anhydride with mixtures of oligomers having at least 12 carbon atoms which are obtainable by oligomerization of a hydrocarbon mixture which comprises at least two olefins having 4 to 8 carbon atoms over a catalyst which comprises a transition metal, processes for the preparation of the alkenylsuccinic anhydrides and their use as sizes in the production of paper, board and cardboard.

The present invention relates to alkenylsuccinic anhydrides obtained from oligomers of C₄- to C₈-olefins and maleic anhydride, processes for their preparation and their use as sizes for paper, board and cardboard.

Hydrocarbon mixtures which comprise short-chain olefins, for example having 2 to 6 carbon atoms, are obtainable on an industrial scale. Thus, for example in the working-up of mineral oil by steam cracking or fluidized catalyst cracking (FCC), a hydrocarbon mixture referred to as C₄-cut and having a high total olefin content is obtained, said olefin content substantially comprising olefins having 4 carbon atoms. Such C₄-cuts, i.e. mixtures of isomeric butenes and butanes, are very suitable, if appropriate after prior removal of the isobutene and hydrogenation of the butadiene present therein, for the preparation of oligomers, in particular of octenes and dodecenes.

The oligomer mixtures which are obtainable from olefin mixtures comprising predominantly linear starting olefins and which are substantially linear have become very important. They are suitable, for example, as a diesel fuel component and as intermediates for the preparation of functionalized, predominantly linear hydrocarbons. Thus, hydroformylation and subsequent hydrogenation of the olefin oligomers give the corresponding alcohols, which are used, inter alia, as starting materials for detergents and as plasticizers. For many fields of use, for example as plasticizer alcohols, the degree of branching of the olefins plays a decisive role. The degree of branching is described, for example, by the ISO index, which indicates the average number of methyl branches of the respective olefin fraction. Thus, for example in the case of a C₈-fraction, the n octenes contribute 0, methylheptenes contribute 1 and dimethylhexenes contribute 2 to the ISO index of the fraction. The lower the ISO index the greater is the linearity of the molecules in the respective fraction.

It is known that catalysts which comprise metals and very predominantly nickel as an active component can be used for the preparation of oligomers which exhibit little branching and are likewise olefinically unsaturated from lower olefins. Heterogeneous catalysts have the advantage over homogeneous ones that isolation of the catalyst from the reactor discharge is dispensed with. Thus, for example, DE-A-43 39 713 (=WO 95/14647) discloses a process for the oligomerization of straight-chained C₂- to C₆-olefins over a fixed-bed catalyst at elevated pressure and elevated temperature, the catalyst comprising, as substantial active constituents, from 10 to 70% by weight of nickel oxide, from 5 to 30% by weight of titanium dioxide and/or zirconium dioxide and from 0 to 20% by weight of alumina and, as the remainder, silica. Further oligomerization catalysts and oligomerization processes are described, for example, in WO 99/25668, WO 00/59849, WO 00/53546, WO 01/72670 and EP-A 1 457 475.

In addition to water so-called oily substances are among the most important raw materials in the formulation of cosmetic and pharmaceutical compositions. WO-A-2004/091555 describes a cosmetic composition which comprises at least one branched α-olefin or one hydrogenation product thereof. The α-olefin has at least one C₂- or longer-chain alkyl branch. It is prepared by oligomerization of certain linear or branched α-olefins in the presence of an acidic catalyst. Disadvantages of these products are their high degree of branching, their content of tert-butyl groups and their nonuniformity, so that the property profile achieved thereby is still worthy of improvement for use in cosmetic and pharmaceutical formulations. Thus, the oligomers used have, for example, a substantial natural odor which is reminiscent of terpenes.

U.S. Pat. No. 3,102,064 discloses the use of aqueous alkenylsuccinic anhydride emulsions which have been stabilized with the aid of cationic starch as engine sizes for paper and paper products.

EP-A 0 593 075 likewise discloses the use of aqueous emulsions of alkenylsuccinic anhydrides which are obtainable by reacting propylene oligomers or n-butylene oligomers with maleic anhydride as sizes.

According to the teaching of EP-A 0 609 879 ethylene oligomers which comprise less than 5% by weight of isomers having 17 or less carbon atoms in the molecule and at least 95% by weight of isomers having at least 18 carbon atoms in the molecule are reacted with maleic anhydride to give alkenylsuccinic anhydrides. The oligomerization of ethylene gives α-olefins, which usually have to be isomerized before the reaction with maleic anhydride so that an internal double bond is present. The reaction products with maleic anhydride which are obtainable therefrom are used as sizes in papermaking. However, the known alkenylsuccinic anhydrides hydrolyze relatively rapidly.

Aqueous size compositions which comprise an emulsified alkenylsuccinic anhydride, a surfactant and a cationic polymer are also known, cf. U.S. Pat. No. 4,657,947 and WO-A 2004/059081.

It is the object of the present invention to provide novel substances which can be used as sizes in papermaking and which have more advantageous performance characteristics than the known alkenylsuccinic anhydrides.

The object is achieved, according to the invention, by alkenylsuccinic anhydrides which can be prepared by reacting oligomers of C₄- to C₈-olefins and maleic anhydride if mixtures of oligomers having at least 12 carbon atoms which are obtainable by oligomerization of a hydrocarbon mixture which comprises at least two olefins having 4 to 8 carbon atoms over a catalyst which comprises a transition metal are used in the reaction with maleic anhydride.

The invention also relates to a process for the preparation of alkenylsuccinic anhydrides by reacting oligomers of C₄- to C₈-olefins and maleic anhydride, mixtures of oligomers having at least 12 carbon atoms which are obtainable by oligomerization of a hydrocarbon mixture which comprises at least two olefins having 4 to 8 carbon atoms over a catalyst which comprises a transition metal being used in the reaction with maleic anhydride.

In order to characterize the oligomers, they can, for example, be completely hydrogenated so that they no longer have any double bonds. Hydrogenation catalysts which may be used are as a rule all catalysts of the prior art which catalyze the hydrogenation of olefins to the corresponding alkanes. The catalysts can be used both in the heterogeneous phase and as homogeneous catalysts. The hydrogenation catalysts preferably comprise at least one metal of group VIII. Particularly suitable metals of group VIII are selected from ruthenium, cobalt, rhodium, nickel, palladium and platinum.

The metals may also be used as mixtures. Moreover, the catalysts may also comprise small amounts of further metals, for example metals of group VIIa, in particular rhenium, or metals of group 1b, i.e. copper, silver or gold, in addition to the metals of group VIII. Particularly preferred metals of group VII are ruthenium, nickel, palladium and platinum, in particular platinum, nickel and palladium, and more preferably palladium and nickel. The catalyst comprises especially palladium as a catalytically active species. The hydrogenation is effected at a temperature of, preferably, from 20 to 250° C., particularly preferably from 50 to 240° C. and in particular from 150 to 220° C. The reaction pressure of the hydrogenation reaction is preferably in the range from 1 to 300 bar, particularly preferably from 50 to 250 bar and in particular from 150 to 230 bar.

In the hydrogenation, an isoalkane mixture whose ¹H-NMR spectrum in the range of a chemical shift δ from 0.6 to 1.0 ppm, based on tetramethylsilane, has an area integral of from 25 to 70%, based on the total integral area, is obtained. It preferably has an area integral of from 30 to 60%, in particular from 35 to 55%, based on the total integral area in the ¹H-NMR spectrum in the range of a chemical shift δ from 0.6 to 1.0 ppm.

Furthermore, the isoalkane mixture preferably has an area integral of up to 95%, particularly preferably up to 98%, based on the total integral area, in the ¹H-NMR spectrum in the range of a chemical shift δ from 0.5 to 3 ppm (i.e. in the range of the aliphatic protons).

Such isoalkane mixtures have substantially no tert-butyl groups (—C(CH₃)₃). The proportion of terminal tert-butyl groups is preferably not more than 20%, particularly preferably not more than 10%, in particular not more than 5% and especially not more than 2%.

The isoalkanes preferably have a uniform structure. Thus, based on the longest continuous carbon chain, they have substantially or exclusively methyl branches. The proportion of side chains having alkyl groups which have 2 or more than two carbon atoms is less than 20%, preferably not more than 10%, particularly preferably not more than 5%, in particular not more than 1%, based on the total number of branching sites.

The isoalkane mixtures preferably comprise from 50 to 90% by weight, in particular from 60 to 80% by weight, of alkanes having 16 carbon atoms and from 0 to 30% by weight, in particular from 10 to 20% by weight, of alkanes having 20 carbon atoms.

The alkane mixture preferably comprises at least 70% by weight, preferably at least 85% by weight, in particular at least 95% by weight, of alkanes having an even number of carbon atoms. A special embodiment is an isoalkane mixture which substantially comprises alkanes having 12 or 16 carbon atoms.

The isoalkane mixtures preferably have a degree of branching B in the range from 0.1 to 0.35, particularly preferably from 0.12 to 0.3, in particular from 0.15 to 0.27 and especially from 0.17 to 0.23.

The products obtainable from the oligomer mixtures by hydrogenation have, for example, a degree of branching B, defined, independently of molecular weight, as the number of branches per carbon atom (B=number of branches/number of carbon atoms, e.g. n-octane: 0/8=0, methyl heptane: 1/8=0.125 , dimethyl hexane: 2/8=0.25, squalane (2,6,10,15,19,23-hexamethyltetracosane): 6/30=0.2.

The oligomers have, for example, preferably from 0.7 to 1.4, in particular from 0.8 to 1.3, CH₃ groups and from 0.7 to 1.3, preferably from 0.8 to 1.2 and in particular from 0.9 to 1.1 olefin groups, in each case per 4 carbon atoms.

Suitable hydrocarbon starting materials for the preparation of olefin oligomers are in principle all compounds which comprise 4 to 8 carbon atoms and at least one ethylenically unsaturated double bond. An industrially available olefin-containing hydrocarbon mixture is preferably used for this purpose.

Preferred industrially available olefin mixtures result from hydrocarbon cracking in mineral oil processing, for example by catalytic cracking, such as fluid catalytic cracking (FCC), thermocracking or hydrocracking with subsequent dehydrogenation. A preferred industrial olefin mixture is the C₄-cut. C₄-cuts are obtainable, for example, by fluid catalytic cracking or steam cracking of gas oil or by steam cracking of naphtha. Depending on the composition of the C₄-cut, a distinction is made between the total C₄-cut (crude C₄-cut), the so-called raffinate I obtained after separating off 1,3-butadiene and the raffinate II obtained after separating off isobutene. A further suitable industrial olefin mixture is the C₅-cut obtainable in naphtha cracking.

Olefin-containing hydrocarbon mixtures suitable for the oligomerization and having 4 to 8 carbon atoms can furthermore be obtained by catalytic dehydrogenation of suitable industrially available paraffin mixtures. Thus, for example, C₄-olefin mixtures can be prepared from liquefied petroleum gases (LPG) and liquefied natural gases (LNG). The latter comprise, in addition to the LPG fraction, also relatively large amounts of higher molecular weight hydrocarbons (light naphtha) and are therefore also suitable for the preparation of C₅- and C₆-olefin mixtures. Olefin-containing hydrocarbon mixtures which comprise monoolefins having 4 to 6 carbon atoms can be prepared from LPG or LNG streams by conventional processes which are known to the person skilled in the art and, in addition to the dehydrogenation, as a rule also comprise one or more working-up steps. These include, for example, the isolation of at least a part of the saturated hydrocarbons present in the abovementioned olefin starting mixtures. These can, for example, be reused for the preparation of olefin starting materials by cracking and/or dehydrogenation. However, the olefins used for the oligomerization may also comprise a proportion of saturated hydrocarbons which are inert to the oligomerization conditions. The proportion of these saturated components is in general not more than 60% by weight, preferably not more than 40% by weight, particularly preferably not more than 20% by weight, based on the total amount of the olefins and saturated hydrocarbons present in the hydrocarbon starting material.

Preferably, a hydrocarbon mixture which comprises from 20 to 100% by weight of C₄-olefins, from 0 to 80% per weight of C₅-olefins, from 0 to 60% by weight of C₆-olefins and from 0 to 10% by weight of olefins differing from the abovementioned olefins, based in each case on the total olefin content, is provided for the oligomerization.

Preferably, a hydrocarbon mixture which has a content of at least 80% by weight, particularly preferably at least 90% by weight and in particular at least 95% by weight, based on the total olefin content, of linear monoolefins is provided for the oligomerization. The linear monoolefins are selected from 1-butene, 2-butene, 1-pentene, 2-pentene, 1-hexene, 2-hexene, 3-hexene and mixtures thereof. It may be advantageous if the hydrocarbon mixture used for the oligomerization comprises up to 20% by weight, preferably up to 5% by weight, in particular up to 3% by weight, of branched olefins, based on the total olefin content.

Particularly preferred oligomers are obtained if a C₄-olefin mixture is used for their preparation. The butene content, based on 1-butene, 2-butene and isobutene, of the C₄-olefin mixture is preferably from 10 to 100% by weight, particularly preferably from 50 to 99% by weight and in particular from 70 to 95% by weight, based on the total olefin content. Preferably, the ratio of 1-butene to 2-butene is in a range from 20:1 to 1:2, in particular from about 10:1 to 1:1. Preferably, the C₄ hydrocarbon mixture comprises less than 5% by weight, in particular less than 3% by weight, of isobutene. Oligomer mixtures are prepared from such mixtures and, for example, trimers, tetramers, pentamers and/or hexamers are isolated therefrom.

The provision of the olefin-containing hydrocarbons may comprise separating off branched olefins. Conventional separation methods which are known from the prior art and are based on different physical properties of linear and branched olefins or on different reactivities which permit the selective reactions are suitable. Thus, for example, isobutene can be separated from C₄-olefin mixtures, such as raffinate I, by one of the following methods:
molecular sieve separation,
fractional distillation,
reversible hydration to give tert-butanol,
acidically catalyzed alcohol addition with a tertiary ether, e.g. methanol addition to give methyl tert-butyl ether (MTBE),
irreversible catalyzed oligomerization to give di- and tri-isobutene,
irreversible polymerization to give polyisobutene.

Such methods are described in K. Weissermel, H.-J. Arpe, Industrielle organische Chemie, 4ₜₕ edition, pages 76-81, VCH-Verlagsgesellschaft Weinheim, 1994, which is hereby incorporated by reference.

In the oligomerization, it is preferable to use a raffinate II which has, for example, the following composition:
from 0.5 to 5% by weight of isobutane,
from 5 to 20% by weight of n-butane,
from 20 to 40% by weight of trans-2-butene,
from 10 to 20% by weight of cis-2-butene,
from 25 to 55% by weight of 1-butene,
from 0.5 to 5% by weight of isobutene
   and trace gases, such as 1,3-butadiene, propene, propane, cyclopropane, propadiene, methyl cyclopropane, vinylacetylene, pentenes, pentanes, etc., in the region of not more than 1% by weight in each case. Tetramers are preferably prepared from the abovementioned mixture.

A suitable raffinate II has, for example, the following typical composition:

| | |
|---|---|
| isobutane and n-butane | 26% by weight |
| isobutene | 1% by weight |
| 1-butene | 26% by weight |
| trans-2-butene | 31% by weight |
| cis-2-butene | 16% by weight. |

It is suitable in particular for the preparation of tetramers. If diolefins or alkynes are present in the olefin-rich hydrocarbon mixture they can be removed therefrom before the oligomerization to preferably less than 10 ppm by weight. They are preferably removed by selective hydrogenation, for example according to EP-A 81 041 and DE-A 15 68 542, particularly preferably by selective hydrogenation to a residual content of less than 5 ppm by weight, in particular 1 ppm by weight.

Oxygen-containing compounds, such as alcohols, aldehydes, ketones or ethers, are also expediently substantially removed from the olefin-rich hydrocarbon mixture. For this purpose, the olefin-rich hydrocarbon mixture can advantageously be passed over an adsorbent, such as, for example, a molecular sieve, in particular one having a pore diameter of from >4 Å to 5 Å. The concentration of oxygen-containing, sulfur-containing, nitrogen-containing and halogen-containing compounds in the olefin-rich hydrocarbon mixture is preferably less than 1 ppm by weight, in particular less than 0.5 ppm by weight.

Catalysts for the Oligomerization

In the context of the present invention, the term “oligomers” comprises dimers, trimers, tetramers and higher products from the synthesis reaction of the olefins used. The oligomers should comprise at least 8 carbon atoms, preferably 12 to 24 carbon atoms, in particular 16 to 20 carbon atoms, in the molecule. The mixtures of oligomers are preferably selected from dimers, in particular from C₆- to C₈-olefins, trimers, in particular from C₄- to C₆-olefins, and tetramers, in particular from C₄-olefins. The mixtures of oligomers are in turn olefinically unsaturated. By a suitable choice of the hydrocarbon material used for the oligomerization and of the oligomerization catalyst, as described below, the desired mixtures of oligomers can be obtained.

A reaction system which comprises one or more, identical or different reactors can be used for the oligomerization. In the simplest case, a single reactor is used for the oligomerization. However, it is also possible to use a plurality of reactors which in each case have identical or different mixing characteristics. The individual reactors can, if desired, be divided once or several times by internals. If two or more reactors form the reaction system, these can be connected to one another in any desired manner, for example in parallel or in series. In a suitable embodiment, for example, a reaction system which consists of two reactors connected in series is used.

Suitable pressure-resistant reaction apparatuses for the oligomerization are known to the person skilled in the art. These include the generally customary reactors for gas-solid and gas-liquid reactions, such as, for example, tubular reactors, stirred kettles, gas circulation reactors, bubble columns, etc. which, if appropriate, can be divided by internals. Tube-bundle reactors or shaft furnaces are preferably used. If a heterogeneous catalyst is used for the oligomerization, it can be arranged in a single fixed catalyst bed or in a plurality of fixed catalyst beds. It is possible to use different catalysts in different reaction zones. However, the use of the same catalyst in all reaction zones is preferred.

The temperature during the oligomerization reaction is in general in a range from about 20 to 280° C., preferably from 25 to 200° C., in particular from 30 to 140° C. The pressure during the oligomerization is in general in a range from about 1 to 300 bar, preferably from 5 to 100 bar and in particular from 20 to 70 bar. If the reaction system comprises more than one reactor, these may have identical or different temperatures and identical or different pressures. Thus, for example, a higher temperature and/or a higher pressure can be established in the second reactor of a reactor cascade than in the first reactor, for example in order to achieve as complete a conversion as possible.

In a special embodiment, the temperature and pressure values used for the oligomerization are chosen so that the olefin-containing starting material is present in liquid form or in the supercritical state.

The oligomerization is preferably carried out adiabatically. In the context of the present invention though this term is understood in the technical sense and not in the physiochemical sense. Thus, the oligomerization reaction takes place as a rule exothermically so that the reaction mixture experiences a temperature increase on flowing through the reaction system, for example a catalyst bed. Adiabatic reaction procedure is understood as meaning a procedure in which the quantity of heat liberated in an exothermic reaction is absorbed by the reaction mixture in the reactor and no cooling by means of cooling apparatuses is used. Thus, the heat of reaction is removed from the reactor with the reaction mixture, apart from a residual proportion which is released to the environment by natural heat conduction and heat radiation from the reactor.

A catalyst comprising transition metals is used for the oligomerization. Heterogeneous catalysts are preferred. Preferred catalysts which are known to result in a low degree of oligomer branching are catalysts comprising nickel. Such catalysts, which are known to result in a low degree of oligomer branching, are generally known to the person skilled in the art. They include the catalysts described in Catalysis Today, 6, 329 (1990), in particular pages 336-338, and those described in DE-A 43 39 713 (=WO-A 95/14647) and DE-A 199 57 173 which are hereby incorporated by reference. A suitable oligomerization process in which the feed stream used for the oligomerization is divided and is fed to at least two reaction zones operated at differing temperatures is described in EP-A 1 457 475, which is likewise incorporated by reference.

The heterogeneous catalysts comprising nickel which are used may have different structures. In principle, unsupported catalysts and supported catalysts are suitable. The latter are preferably used. The support materials may be, for example, silica, alumina, aluminosilicates, aluminosilicates having sheet structures and zeolites, such as mordenite, faujasite, zeolite X, zeolite Y and ZSM-5, zirconium oxide which is treated with acids, or sulfated titanium dioxide. Particularly suitable are precipitated catalysts which are obtainable by mixing of aqueous solutions of nickel salts and silicates, e.g. sodium silicate with nickel nitrate, and, if appropriate, aluminum salts, such as aluminum nitrate and calcination. Catalysts which are obtained by incorporating Ni²⁺ ions by ion exchange into natural or synthetic sheet silicates, such as montmorillonites, may furthermore be used. Suitable catalysts can also be obtained by impregnation of silica, alumina or aluminosilicates with aqueous solutions of soluble nickel salts, such as nickel nitrate, nickel sulfate or nickel chloride, and subsequent calcination.

Catalysts comprising nickel oxide are preferred. Catalysts which substantially comprise NiO, SiO₂, TiO₂ and/or ZrO₂ and, if appropriate, Al₂O₃ are particularly preferred. Most preferred is a catalyst which comprises from 10 to 70% by weight of nickel oxide, from 5 to 30% by weight of titanium dioxide and/or zirconium dioxide, and from 0 to 20% by weight of alumina as essential active constituents, the remainder comprising silica. Such a catalyst is obtainable, for example, by precipitation of a catalyst material at pH 5 to 9 by addition of an aqueous solution comprising nickel nitrate to give an alkali-waterglass solution which comprises titanium dioxide and/or zirconium dioxide, filtration, drying and heating at from 350 to 650° C. For the preparation of these catalysts, reference is made specifically to DE-A 43 39 713. The disclosure of this publication and the prior art cited therein are hereby incorporated by reference.

In a further embodiment, a nickel catalyst according to DE-A 199 57 173 is used as a catalyst for the oligomerization. This is substantially alumina which was treated with a nickel compound and a sulfur compound. A molar ratio of sulfur to nickel in the range from 0.25:1 to 0.38:1 is preferably present in the prepared catalyst.

The catalyst is preferably present in the form of pieces, for example in the form of tablets, e.g. having a diameter from 2 to 6 mm and a height from 3 to 5 mm, rings having, for example, an external diameter of from 5 to 7 mm, a height from 2 to 5 mm and a hole diameter of from 2 to 3 mm, or extrudates of different length which have a diameter of, for example, from 1.5 to 5 mm. Such forms are obtained in a manner known per se by tabletting or extrusion, generally using a tabletting assistant, such as graphite or stearic acid.

For separation, the reaction mixture of the oligomerization can be subjected to one or more separation steps. Suitable separation apparatuses are the conventional apparatuses known to the person skilled in the art. These include, for example, distillation columns, e.g. tray columns, which, if desired, can be equipped with bubble caps, perforated plates, sieve trays, valves, side take-offs, etc., evaporators, such as thin-film evaporators, falling-film evaporators, wiped-surface evaporators, Sambay evaporators etc. and combinations thereof. The isolation of the olefin fraction is preferably effected by single-stage or multi stage fractional distillation.

Reaction of the Oligomers with Maleic Anhydride

The mixtures of oligomers described above are reacted with maleic an hydride in analogy to known processes. Mixtures of alkenylsuccinic anhydrides form. Saturated hydrocarbons, which, if appropriate, are present in the mixture of the oligomers, do not as a rule interfere with the reaction with maleic anhydride. However, they can also be removed from the oligomer mixture before the reaction, for example by distillation. The reaction with maleic anhydride is preferably effected in the absence of a solvent at a temperature in the range of, for example, from 100 to 280° C., preferably at from 150 to 250° C., generally at from 180 to 230° C. The reaction is preferably carried out in pressure-resistant apparatuses, such as autoclaves. It can be effected batchwise or continuously. The residence time of the reaction mixture in the reaction zone depends on the reaction temperature chosen in each case. Higher temperatures require shorter reaction times than lower temperatures. Thus, the reaction time may be, for example from 5 seconds to 10 hours. For example, from 0.2 to 5 mol, preferably from 0.3 to 3 mol and in particular from 0.8 to 2.4 mol of maleic anhydride are used per mole of oligomer in the mixture of oligomers. The mixtures of alkenylsuccinic esters forming in the reaction can be used without an additional purification step as sizes for paper. However, low-boiling fractions can also be distilled off from the reaction mixture beforehand or, in an advantageous embodiment of the invention, the reaction mixture can be subjected to fractional distillation. The main amount of the mixture of alkenylsuccinic anhydrides is distilled off from the mixture under a pressure of 1 mbar in the temperature range from 180 to 240° C. In general, the distillation is terminated when the vapors passing over under a pressure of 1 mbar have a temperature of 230° C.

Use of the Alkenylsuccinic Anhydrides

The invention also relates to the use of the above-described mixtures of alkenylsuccinic anhydrides as sizes for paper. For this purpose, the mixtures of the alkenylsuccinic anhydrides are emulsified in water in the presence of at least one protective colloid. Suitable protective colloids are, for example, all types of starch, for example both amylose and amylopectin, natural starches, hydrophobically or hydrophilically modified starches, degraded starches, it being possible for the starch degradation to be carried out, for example, oxidatively, thermally, hydrolytically or enzymatically and for both natural and modified starches to be used for the starch degradation, dextrins and crosslinked, water-soluble starches, cf. Ullmanns Encyclopedia of Industrial Chemistry, 6^{th} edition, volume 33, under Starch, pages 735 to 737. Conventional crosslinking agents for the preparation of such starches are, for example, POCl₃, epichlorohydrin and mixed anhydrides. Further examples of protective colloids are glycogens, inulins, chitins, chitosans, pectins, water-soluble cellulose derivatives, such as carboxyalkylcelluloses, cellulose sulfate, cellulose phosphoric acid esters, cellulose formate, hydroxyethylcelluloses, hemicelluloses, such as xylans, mannans, galactans, glycoproteins and mucopolysaccharides.

Natural starches that can be converted into a water-soluble form, for example, with the aid of a starch digestion, cationic starch, preferably cationically modified potato starch and anionically modified starches, such as oxidized potato starch are preferably used as the protective colloid. The preferably used protective colloids also include anionically modified starches which were subjected to a decrease in molecular weight. The decrease in molecular weight is preferably brought about enzymatically. The average molar mass of the degraded starches is, for example, from 500 to 100 000, in general from 1000 to 30 000. Suitable degraded starches are described, for example, in EP-A 0 257 412 and in EP-A 0 276 770.

Other suitable protective colloids are condensates of
naphthalenesulfonic acid and formaldehyde,
phenol, phenolsulfonic acid and formaldehyde,
naphthalenesulfonic acid, formaldehyde and urea and
phenol, phenolsulfonic acid, formaldehyde and urea.

These are known compounds which can be prepared, for example, by condensation of the abovementioned constituents in the presence of acids, such as sulfuric acid or p-toluenesulfonic acid, as a catalyst. Instead of the free acids, it is also possible to use the salts of naphthalenesulfonic acid or of phenolsulfonic acid in the condensation. The molar ratio of the last-mentioned acids to formaldehyde in the condensation is, for example, from 1:0.1 to 1:2, in general from 1:0.5 to 1:1. If the condensation of naphthalenesulfonic acid and of phenolsulfonic acid with formaldehyde is additionally carried out in the presence of urea, for example from 0.1 to 5 mol of urea, based on one mole of the mixture of phenol and phenolsulfonic acid or on 1 mol of naphthalenesulfonic acid, are used.

Further suitable protective colloids are polymers of ethylenically unsaturated C₃- to C₅-carboxylic acids, in particular polymers of acrylic acid, and amphiphilic copolymers of (i) hydrophobic monoethylenically unsaturated monomers and (ii) monoethylenically unsaturated carboxylic acids, monoethylenically unsaturated sulfonic acids, monoethylenically unsaturated phosphonic acids or mixtures thereof. Copolymers of (i) styrene, isobutene and/or diisobutene and (ii) of an ethylenically unsaturated C₃- to C₅-carboxylic acid, such as acrylic acid, maleic acid and/or methacrylic acid are an example of such protective colloids. The anionic protective colloids may be used in the form of the free acids and in partly or completely neutralized form. Suitable neutralizing agents are, for example, alkalis, ammonia and amines and alkaline earth metal bases. In general, sodium hydroxide solution, sodium carbonate, sodium bicarbonate, ammonia, triethanolamine, morpholine, magnesium oxide or calcium hydroxide is used for the neutralization. The molar mass M_{w} of the amphiphilic copolymers and of the homopolymers of the ethylenically unsaturated carboxylic acid is, for example, in the range from 500 to 100 000, preferably from 1000 to 10 000.

In principle, all surface-active compounds or polymers which, according to the prior art, are present in alkenylsuccinic anhydride emulsions can be used as protective colloids, cf. U.S. Pat. No. 4,657,946 and WO-A 2004/059081. Thus, the cationic polymers disclosed in U.S. Pat. No. 4,657,946 and having molar masses of from 10 000 to less than 1 million in combination with surfactants are particularly suitable. Suitable cationic polymers are diallyldimethylammonium chloride, basic acrylates and basic methacrylates in the form of the free bases or of the quaternized products, e.g. dimethylaminoethyl methacrylate, dimethylaminoethyl acrylate, diethylaminoethyl acrylate, diethylaminoethyl methacrylate and the corresponding quaternized products. Quaternizing agents which may be used are, for example, methyl chloride, ethyl chloride, hexyl chloride, benzyl chloride or dimethyl sulfate. Other suitable cationic polymers are Mannich products of acrylamide, formaldehyde and a secondary amine and quaternized Mannich products.

Preferred polymers from this group are diallyldimethylammonium chloride and basic (meth)acrylates in the form of the salts or of the quaternized compounds. Cationic polymers which are suitable as the protective colloid for alkenylsuccinic anhydrides are described in detail in U.S. Pat. No. 4,657,946, column 3, line 56 to column 5, line 36.

Examples of surface-active compounds are to be found in this reference in column 2, line 57 to column 3, line 55.

Customary surfactants for alkenylsuccinic anhydrides are described, for example, in WO-A 2004/059081, cited in the prior art, page 11, line 5 to page 12, line 16 and in the table on page 31. These are, for example, sulfosuccinates, alkyl- and arylamides, primary, secondary and tertiary amines and the corresponding quaternary salts, ethoxylated fatty acids, fatty alcohols, ethoxylated fatty alcohols, fatty acid esters, ethoxylated fatty acid esters, phosphate esters, polyethylene glycols, alkanesulfonates, arylsulfonates, arylsulfates and alkyl sulfates.

Frequently used emulsifiers are surface-active substances, such as sodium C₁₂- to C₂₂-alkanesulfonates or polyvinyl alcohol.

The anionic protective colloids are used, for example, in an amount of from 0.05 to 20, preferably from 0.5 to 10, % by weight, based on the mixture of alkenylsuccinic anhydrides, during the emulsification. The amphiphilic polymers are preferably used in an amount of from 0.1 to 2% by weight, based on the mixture of alkenylsuccinic anhydrides. In the case of the protective colloids based on starch and derivatives thereof, for example, from 1 to 10, preferably from 2 to 4, parts by weight of starch or derivatives thereof are used per 1 part by weight of the mixture of alkenylsuccinic anhydrides.

The mixtures of alkenylsuccinic anhydrides are emulsified by known methods under the action of shear forces in water and in the presence of at least one protective colloid. The emulsification is effected, for example, with the aid of high-pressure homogenizers, or rotor-stator apparatuses or by the action of ultrasound. Information on suitable apparatuses can be found, for example, in the publication by H. Schubert et.al., Mischen und Rühren-Grundlagen und moderne Verfahren für die Praxis, VDI-Tagung, Nov. 23-24, 1988, Baden-Baden, under Neue Entwicklungen auf dem Gebiet der Emulgiertechnik. The emulsification is effected, for example, in the temperature range from 0 to 100, in general from 20 to 60, ° C.

The preparation of the emulsions is effected as a rule a short time before use because the alkenylsuccinic anhydrides (ASA) hydrolyze in the presence of water. In general, concentrated aqueous emulsions of ASA are first prepared (for example, the ASA concentration is up to 50% by weight, preferably from 10 to 20% by weight, and the concentrated ASA emulsions are then diluted to an ASA content of, for example, from 0.7 to 1.2% by weight, preferably about 1% by weight. The dilute ASA emulsions are then used as size for paper.

Of particular technical interest is the use of a mixture of alkenylsuccinic anhydrides which is obtainable by
(i) oligomerization of a hydrocarbon mixture which comprises
   from 0.5 to 5% by weight of isobutane,
   from 5 to 20% by weight of n-butane,
   from 20 to 40% by weight of trans-2-butene,
   from 10 to 20% by weight of cis-2-butene,
   from 25 to 55% by weight of 1-butene and
   from 0.5 to 5% by weight of isobutene
to give a mixture of oligomers which comprises isobutane and n-butane and
(ii) reaction of this mixture with maleic anhydride to give a mixture of C₁₆- to C₂₄-alkenylsuccinic anhydrides
   as size for paper, board and cardboard. Such sizes can be prepared more simply than the reaction products of an n-butene oligomer with maleic anhydride which are known from the prior art, because, for example, the butene mixture can react directly with maleic anhydride without prior isomerization. Owing to the high degree of branching of the olefin mixture reaction products with maleic anhydride which have a low melting point and which are liquid, for example at room temperature, and therefore easier to handle are obtained.

The alkenylsuccinic anhydrides are preferably used as engine size in papermaking but can also be used as surface size. The amounts of size added to the paper stock are, for example, from 0.1 to 2, preferably from 0.5 to 1.0 kg/t of dry paper.

For the production of paper, board and cardboard, it is possible to start from cellulose fibers of all types, both from natural and recovered fibers, in particular from fibers obtained from wastepaper. Suitable fibers for the production of the pulps are all qualities customary for this purpose, e.g. mechanical pulp, bleached and unbleached chemical pulp and paper stocks from all annual plants. Mechanical pulp includes, for example, groundwood, thermomechanical pulp (TMP), chemothermomechanical pulp (CTMP), pressure groundwood, semichemical pulp, high-yield chemical pulp and refiner mechanical pulp (RMP). For example sulfate, sulfite and soda pulps are suitable as chemical pulp. Unbleached pulp, which is also referred to as unbleached kraft pulp, is preferably used. Suitable annual plants for the production of paper stocks are, for example, rice, wheat, sugar cane and kenaf. The pulps can also advantageously be produced using wastepaper, which is used either alone or as a mixture with other fibers, or fiber mixtures comprising a primary stock and recycled coated broke are used as starting material, for example bleached pine sulfate mixed with recycled coated broke.

The alkenylsuccinic anhydrides are preferably used for the sizing of paper products for the production of liquid packaging and of paper products which are required in the building sector, for example sized papers or cardboard for sandwich-type plasterboards. They can also be used as sizes in the production of liners, wood-containing and wood-free printing and writing papers and recycled papers.

The alkenylsuccinic anhydrides can also as curing agents for epoxy resins, as additives for fuels and lubricants (e.g. as dispersants) as rust and corrosion inhibitors, as surfactants, as dispersants, in particular in mineral oil production, as food additives, and for imparting water repellency to leather and textiles. The alkenylsuccinic anhydrides can furthermore be converted into amide, imide and ester derivatives and into alkenylsuccinic acid and then used as dispersants, as surfactants, as additives for lubricating oils and as corrosion inhibitors.

The parts stated in the examples are parts by weight and the percentages stated are percent by weight, unless otherwise evident from the context. The determination of the Cobb value was effected according to DIN 53 132 by storage of the paper sheets for a period of 60 seconds in water. The water absorption is stated in g/m². The ink flotation time was determined according to DIN 53126 using a blue test ink.

### EXAMPLES

The following hydrocarbon mixture (raffinate II) was used for the preparation of a mixture of oligomers:
5 parts of isobutane,
16 parts of n-butane,
31 parts of 1-butene,
28 parts of trans-2-butene,
15 parts of cis-2-butene and
2 parts of isobutene.

The catalyst used was a material which was shaped according to DE 4339713 to give 5.5 mm solid tablets (composition in % by weight, 50% of NiO, 12.5% of TiO₂, 33.5% of SiO₂, 4% of Al₂O₃).

The experiments were carried out in a reactor cascade consisting of two reactors connected in series (diameter 80 mm, length 4000 mm, intermediate cooling between the two reactors) with subsequent distillation column. A mixture of raffinate II according to the above composition was fed to the reactor entrance of the first reactor under reaction conditions. In addition, a circulating stream (reactor exit stream from the second reactor) was recycled directly to the reactor entrance.

The catalyst was introduced into both reactors and dried for 24 h while passing through 30 m³(STP)/h of N₂ at atmospheric pressure and at a reactor temperature of 170° C. The catalyst was then operated under the following conditions: raffinate II feed (10 kg/h), circulation (50 kg/h) and pressure (30 bar) and temperature (50° C.). Under these conditions, a C₄-olefin conversion of 55% was achieved and a C₈₊-olefin discharge which consisted of 70% of butene dimers, 22% of butene trimers, 7% of butene tetramers and 1% of C20+ olefins resulted. The C₈₊ discharge was distilled. An oligomer mixture which consisted of 7% of butene trimers, 70% of butene tetramers, 17% of butene pentamers and 5% of butene hexamers and 1% of butene heptamers was obtained (referred to below as “butene oligomer mixture”).

### Examples 1 to 6

Preparation of adducts from butene oligomer mixture and maleic anhydride In each case the amounts shown in table 1 of butene oligomer mixture and 98 g (1 mol) of maleic anhydride (MAA) were initially taken under a nitrogen atmosphere in a 1.2 | autoclave, heated to a temperature of 220° C., by means of a metal bath after the autoclave had been closed and stirred at this temperature for 5 hours. The pressure increased to 1.2-1.3 bar. After 5 hours, the autoclave was cooled. A brown, low-viscosity liquid was obtained. The low-boiling fractions were removed in a rotary evaporator at 10 mbar and an internal temperature up to 180° C. A brown, odorless, viscous liquid was obtained. The molar ratios and product weights are shown in table 1.

TABLE 1

| | | Molar ratio of | |
|---|---|---|---|
| | Amount of butene | butene oligomer | |
| Example | oligomer mixture | mixture:MAA | Amountisolated |
| 1 | 233 g | ~1:1 | 249 g (75%) |
| 2 | 256 g | ~1.1:1 | 258 g (73%) |
| 3 | 303 g | ~1.3:1 | 278 g (69%) |
| 4 | 373 g | ~1.6:1 | 295 g (63%) |
| 5 | 419 g | ~1.8:1 | 318 g (61%) |
| 6 | 466 g | ~2:1 | 328 g (58%) |

### Examples 7 to 9

Preparation of Adducts of Butene Oligomer Mixture and Maleic Anhydride at Various Temperatures

224 g of a butene oligomer mixture and 98 g (1 mol) of maleic anhydride (MAA) were initially taken under nitrogen in a 1.2 l autoclave, heated to the temperature stated in each case in table 2 in a metal bath after the autoclave had been closed and stirred at this temperature for 5 hours. The autoclave was then cooled. A brown low-viscosity liquid was obtained. The low-boiling fractions were removed in a rotary evaporator at 1 mbar and an internal temperature up to 160° C. A brown, odorless, viscous liquid was obtained. The product weights are shown in table 2.

TABLE 2

| | Pressure/ | | | |
|---|---|---|---|---|
| Example | bar | Temperature/° C. | | Amount isolated |
| 7 | 1.0 | 200 | | 182 g (56%) |
| 8 | 1.3 | 220 | | 243 g (75%) |
| 9 | 6.0 | 250° | C. | 255 g (79%) |

### Examples 10 to 14

Preparation of Adducts of Butene Oligomer Mixture and Maleic Anhydride in Various Reaction Times

233 g of butene oligomer mixture and 98 g (1 mol) of maleic anhydride (MAA) were initially taken under a nitrogen atmosphere in a 1.2 l autoclave, heated to a temperature of 220° C. by means of a metal bath after the autoclave had been closed and stirred at this temperature. After the time stated in each case in table 3, the reaction was stopped by cooling the autoclave and the reaction mixture was isolated. A brown slightly viscous liquid was obtained. The low-boiling fractions were removed by heating the reaction mixture in a rotary evaporator at 10 mbar to a temperature of 180° C. In each case a brown, odorless, viscous liquid was obtained. The product weights (amounts isolated) are shown in table 3.

TABLE 3

| | Pressure/ | Time/ | |
|---|---|---|---|
| Example | bar | hours | Amount isolated |
| 10 | 1.3 | 1 | 194 g (59%) |
| 11 | 1.3 | 4 | 240 g (73%) |
| 12 | 1.2 | 5 | 249 g (75%) |
| 13 | 1.3 | 6 | 257 g (78%) |
| 14 | 1.7 | 8 | 273 g (82%) |

### Example 15

Distillation of an Adduct of MAA with a Butene Oligomer Mixture

240 g of that adduct of MAA with butene oligomer mixture which was prepared according to example 8 was distilled in a 500 ml flask over a 40 cm column at a pressure of 1 mbar. The heat transfer medium used was a metal bath. 3 fractions and a forerun were collected up to a distillation temperature of 230° C. The bottom product was a black, highly viscous liquid. The forerun and the three fractions were yellow, slightly viscous liquids. Distillation data are shown in table 4.

TABLE 4

| | Bath | | |
|---|---|---|---|
| | temperature/ | Distillation | |
| Fractions | ° C. | temperature/° C. | Amount isolated/g |
| Forerun | 180 | 142 | 5 g (2%) |
| Fraction 1 | 200 | 160 | 81 g (34%) |
| Fraction 2 | 215 | 190 | 68 g (28%) |
| Fraction 3 | 270 | 230 | 21 g (9%) |
| Bottom | — | | 65 g (27%) |
| product | | | |

### Example 16

Distillation of the Butene Oligomer Mixture

3 l (2360 g) of the butene oligomer mixture described above were distilled in a 4 l flask over a bridge at a pressure of 10 mbar. The heat transfer medium used was an oil bath. Altogether, 1711 g of a butene oligomer mixture were collected as distillate up to a distillation temperature of 180° C. It consisted of 10% of butene trimers, 78% of butene tetramers, 10% of butene pentamers and 2% of butene hexamers.

### Example 17

Preparation of Adducts from a Distillate of Butene Oligomer Mixture (Obtained According to Example 16)

224 g of distillate of butene oligomer mixture according to example 16 and 98 g (1 mol) of maleic anhydride (MAA) were initially taken under a nitrogen atmosphere in a 1.2 l autoclave, heated to a temperature of 220° C. by means of a metal bath after the autoclave had been closed and stirred at this temperature for 5 hours. After 5 hours, the reaction was stopped by cooling the autoclave and the reaction mixture was isolated. A brown slightly viscous liquid was obtained. The low-boiling fractions were removed by heating the reaction mixture in a rotary evaporator at 10 mbar to a temperature of 180° C. In each case a brown, odorless, viscous liquid was obtained. The product weight was 261 g (81%).

### Example 18

Preparation of Adducts from a Distillate of Butene Oligomer Mixture

336 g of a pure C₁₆ butene oligomer mixture (100% C₁₆ olefins) and 98 g (1 mol) of maleic anhydride (MAA) were initially taken under a nitrogen atmosphere in a 1.2 l autoclave, heated to a temperature of 210° C. by means of a metal bath after the autoclave had been closed and stirred at this temperature for 5 hours. After 5 hours, the reaction was stopped by cooling the autoclave and the reaction mixture was isolated. A brown slightly viscous liquid was obtained. The low-boiling fractions were removed by heating the reaction mixture in a rotary evaporator at 1 mbar to a temperature of 180° C. In each case, a brown, odorless, viscous liquid was obtained. The product weight was 192.7 g and the product contained only alkenylsuccinic anhydride.

17.8 g of a residual MAA and 223.5 g of a residual olefin were separated off as byproducts.

Use Examples

Preparation of ASA Emulsions

A 5% strength suspension of a cationized starch (Hicat® 5163A, from Roquette) was refluxed for 30 minutes in a flask with a mechanical stirrer until the starch had dissolved without leaving a residue. Thereafter, the starch solution was cooled to room temperature in an ice bath and adjusted to pH 4 with formic acid (1% in water).

For the preparation of the ASA emulsion, 200 g of the starch solution were transferred to an upright mixer with a glass jug (from ABC Elektro, Model 260) and in each case 2 g of the MAA/olefin adduct (ASA) stated in table 5 were added. The emulsification was effected for 30 seconds at full power and for 90 seconds at half power. The particle size distribution was measured on an apparatus from Coulter, Model LS130. The results of the emulsification experiments are summarized in table 5:

TABLE 5

| | MAA/olefin adduct (ASA) | |
|---|---|---|
| Example | prepared according to | D50 [μm] |
| 19 | Example 1 | 1.6 |
| 20 | Example 3 | 1.4 |
| 21 | Example 4 | 1.2 |
| 22 | Example 6 | 1.1 |
| 23 | Example 18 | 1.5 |

Use of the Emulsions for the Sizing of Paper and Cardboard

In a first experimental series a chemical pulp suspension consisting of birch and pine sulfate was prepared. 20% of ground calcium carbonate and 0.6% of a cationic wet end starch were added to said suspension. The ASA emulsions described above were then added. After addition of a retention aid based on polyacrylamide, in each case sheets were produced by means of a Rapid-Köthen sheet former. The sheets thus produced were dried on a drying cylinder. The measurement was carried out after conditioning at 50% humidity for 24 h.

TABLE 6

| | Amount of | | |
|---|---|---|---|
| ASA emulsion | ASA metered | Cobb 60″ | Ink flotation time |
| according to | [kg/t] | [g/m²] | [min] |
| Example 19 | 2 | 32 | 10 |
| Example 20 | 2 | 27 | 11 |
| Example 21 | 2 | 25 | 15 |
| Example 22 | 2 | 24 | 19 |
| Example 23 | 2 | 29 | 25 |

In a further experimental series, a stock suspension which consisted of 100% of wastepaper was prepared. 0.8% of a cationic wet end starch was first added to said stock suspension. The ASA dispersions described above, which were prepared according to examples 19-22, were then added. After addition of a retention aid based on polyacrylamide, in each case sheets were produced by means of a Rapid-Köthen sheet former. The sheets thus produced were dried at 90° C. on a drying cylinder and then conditioned at 50% humidity for 24 h. The sizing was then determined according to ink flotation time and Cobb 60. The results are shown in table 7.

TABLE 7

| | Amount of ASA | | |
|---|---|---|---|
| ASA emulsion | metered | Cobb 60″ | Ink flotation time |
| according to | [kg/t] | [g/m²] | [min] |
| Example 19 | 3 | 41 | 23 |
| Example 20 | 3 | 35 | 27 |
| Example 21 | 3 | 35 | 33 |
| Example 22 | 3 | 34 | 38 |

In a further experimental series, a chemical pulp suspension consisting of bleached birch sulfate and pine sulfate was prepared. 0.75% of a cationic wet end starch was first added to said suspension. The ASA emulsions described in examples 19-22 were then added. After addition of a retention aid based on polyacrylamide, in each case sheets having a basis weight of 150 g/m² were produced by means of a Rapid-Köthen sheet former. The sheets thus produced were dried at 90° C. on a drying cylinder and then conditioned at 50% humidity for 24 h. An adhesive tape was then applied to both sides of the sheets without stripes. Strips having a length of 25×75 mm were cut from the sheets. The test strips were immersed in a hydrogen peroxide bath at 70° C. in order to determine the edge penetration by differential weighing. The results are shown in table 8.

TABLE 8

| | Amount of ASA | Peroxide edge |
|---|---|---|
| ASA emulsion | metered | penetration, 70° C. |
| according to | [kg/t] | [kg/m²] |
| Example 19 | 3 | 3.25 |
| Example 20 | 3 | 3.30 |
| Example 21 | 3 | 2.70 |
| Example 22 | 3 | 2.25 |

1. An alkenylsuccinic anhydride which can be prepared by reacting oligomers of C₄-C₈-olefins and maleic anhydride,
wherein a mixture of oligomers having at least 12 carbon atoms which is obtainable by an oligomerization of a hydrocarbon mixture, which comprises at least two olefins having 4 to 8 carbon atoms, over a catalyst which comprises a transition metal is used in the reaction with maleic anhydride, and
a hydrocarbon mixture which comprises from 20 to 100% by weight of C₄-olefins, from 0 to 80% by weight of C₅-olefins, from 0 to 60% by weight of C₆-olefins and from 0 to 10% by weight of olefins differing from the abovementioned olefins, based in each case on the total olefin content, is used in the oligomerization.

2. The alkenylsuccinic anhydride according to claim 1, wherein a hydrocarbon mixture which has a content of at least 80% by weight, based on the total olefin content, of linear monoolefins is used in the oligomerization.

3. The alkenylsuccinic anhydride according to claim 1, wherein a C₄-olefin mixture is used in the oligomerization.

4. The alkenylsuccinic anhydride according to claim 3, wherein a C₄-olefin mixture which comprises 1-butene and 2-butene in a weight ratio of from 20:1 to 1:2 is used in the oligomerization and trimers, tetramers, pentamers and/or hexamers are isolated from the oligomer mixture.

5. The alkenylsuccinic anhydride according to claim 1, wherein a hydrocarbon mixture which comprises
from 0.5 to 5% by weight of isobutane,
from 5 to 20% by weight of n-butane,
from 20 to 40% by weight of trans-2-butene,
from 10 to 20% by weight of cis-2-butene,
from 25 to 55% by weight of 1-butene and
from 0.5 to 5% by weight of isobutene
is used in the oligomerization.

6. A process for the preparation of alkenylsuccinic anhydrides by reacting oligomers of C₄- to C₈-olefins and maleic anhydride,
wherein a mixture of oligomers having at least 12 carbon atoms is obtainable by an oligomerization of a hydrocarbon mixture, which comprises at least two olefins having 4 to 8 carbon atoms, over a catalyst which comprises a transition metal is used in the reaction with maleic anhydride, and
a hydrocarbon mixture which comprises from 20 to 100% by weight of C₄-olefins, from 0 to 80% by weight of C₅-olefins, from 0 to 60% by weight of C₆-olefins and from 0 to 10% by weight of olefins differing from the abovementioned olefins, based in each case on the total olefin content, is used in the oligomerization.

7. A process for sizing paper, comprising emulsifying a mixture which comprises pulp and alkenylsuccinic anhydrides according to claim 1.

8. The process according to claim 7, wherein a mixture of alkenylsuccinic anhydrides is used which is obtainable by
(i) oligomerization of a hydrocarbon mixture which comprises
from 0.5 to 5% by weight of isobutane,
from 5 to 20% by weight of n-butane,
from 20 to 40% by weight of trans-2-butene,
from 10 to 20% by weight of cis-2-butene,
from 25 to 55% by weight of 1-butene and
from 0.5 to 5% by weight of isobutene
to give a mixture of butene oligomers which comprises n-butane and isobutane and
(ii) reaction of this mixture with maleic anhydride to give a mixture of C₁₆- to C₂₄-alkenylsuccinic anhydrides.

9. The process according to claim 6, wherein the catalyst comprises from 10 to 70 wt % of NiO, from 5 to 30 wt % of TiO₂ and, and from 0 to 20 wt % of Al₂O₃.

10. The process according to claim 6, wherein the oligomerization occurs at a temperature ranging from 20 to 280° C.
